SEQUENCE LISTING (2 sequences)

SEQ ID NO: 1 - protein, 21 aa, synthetic construct
  source 1..21: note = Amino acid sequence of a polypeptide fragment THRB-CVD20
CVATNAQGSHWKQKRKFLPED

SEQ ID NO: 2 - protein, 15 aa, synthetic construct
  source 1..15: note = Amino acid sequence of polypeptide fragment in the comparative example
CKSIGHKPEPTDEEW

(19) United States
(12) Patent Application Publication
(10) Pub. No.: US 2025/0313609 A1
(43) Pub. Date: Oct. 9, 2025

(54) Polypeptide Fragment Thyroid Hormone Receptor BETA1 (THRB)-CVD20, and Polyclonal Antibody Prepared Using the Same and Use Thereof

(71) Applicant: Beijing Biosynthesis Biotechnology Co., Ltd., Beijing (CN)

(72) Inventors: Zhijuan JI, Beijing (CN); Ju WU, Beijing (CN); Fengqiang ZHAO, Beijing (CN)

(21) Appl. No.: 18/626,298

(22) Filed: Apr. 3, 2024

(51) Int. Cl. C07K 14/72; C07K 1/06; C07K 1/08; C07K 16/28

(52) U.S. Cl. CPC C07K 14/721; C07K 1/061; C07K 1/088; C07K 16/2869

(57) ABSTRACT

The present disclosure relates to the technical field of antibody engineering, and specifically provides a polypeptide fragment thyroid hormone receptor betal (THRB)-CVD20, and a polyclonal antibody prepared using the same and use thereof. The present disclosure provides a polypeptide fragment THRB-CVD20, wherein the polypeptide fragment has an amino acid sequence shown in SEQ ID NO: 1. The present disclosure further provides a preparation method of the polypeptide fragment and a polyclonal antibody prepared using the polypeptide fragment. In the present disclosure, the polyclonal antibody can accurately identify target cells due to high sensitivity and specificity, and can be further used to identify, identify, and screen the THRB.

### REFERENCE TO SEQUENCE LISTING

A computer readable XML file entitled “GWP20240100833”, that was created on Apr. 2, 2024, with a file size of about 2916 bytes, contains the sequence listing for this application, has been filed with this application, and is hereby incorporated by reference in its entirety.

### TECHNICAL FIELD

The present disclosure relates to the technical field of antibody engineering, and specifically relates to a polypeptide fragment thyroid hormone receptor betal (THRB)-CVD20, and a polyclonal antibody prepared using the same and use thereof.

### BACKGROUND

Thyroid hormone plays an important role in the growth and development balance of mammals. This hormone regulates a metabolic rate of genes related to the intestine, bones, heart muscle, liver, and central nervous system of mammals, cholesterol and triglyceride levels, as well as heart rate, and can even affect mood and overall well-being.

Thyroid hormone receptors (TRs) are nuclear receptors that belong to the superfamily of eukaryotic transcription factors. By binding to the TRs, thyroid hormone acts on the transcription to achieve positive/negative regulation of target genes, playing an extremely important role in the normal differentiation, development, and metabolic balance of the body. TRs include two subtypes, alpha receptors and beta receptors. Among them, the subtype thyroid hormone receptor betal (THRB) can play a tumor suppressor function through transcriptional regulation and thus shows a strong and important role in the liver and other tissues. Therefore, THRB has become an important target for drug research and gene therapy. The recognition, identification, and screening of THRB can help further elucidate the important functions of THRB in tumorigenesis and treatment.

Currently, most commercial THRB antibodies are not suitable for flow cytometric sorting applications and cannot meet the demands of scientists to recognize, identify, and screen specific THRB-positive living cells, thus limiting in-depth research on the THRB functions.

### SUMMARY

A purpose of the present disclosure is to further improve the sensitivity and specificity of THRB antibodies to accurately locate and identify target cells. The present disclosure provides a polypeptide fragment THRB-CVD20, and a polyclonal antibody prepared using the same and use thereof. The polyclonal antibody can be applied to flow cytometric sorting, thereby enabling the recognition, identification, and screening of THRB-positive living cells.

In the first aspect, the present disclosure provides a polypeptide fragment THRB-CVD20, adopting the following technical solutions:

The present disclosure provides a polypeptide fragment THRB-CVD20, where the polypeptide fragment THRB-CVD20 has an amino acid sequence shown in SEQ ID NO: 1.

In the second aspect, the present disclosure further provides a preparation method of the polypeptide fragment THRB-CVD20, including the following steps: resin swelling, amino acid activation, preparation of an amino acid-resin, removal of a protecting group, preparation of a peptide resin, and separation and purification.

In the present disclosure, the polypeptide fragment THRB-CVD20 with high purity and yield is prepared by the resin swelling, amino acid activation, preparation of an amino acid-resin, removal of a protecting group, preparation of a peptide resin, and separation and purification according to the amino acid sequence shown in SEQ ID NO: 1.

Preferably, a solvent for the resin swelling is selected from the group consisting of N-methylpyrrolidone (NMP), N,N-dimethylformamide (DMF), and dichloromethane (DCM).

Through experimental analysis, it can be seen that compared to using the DMF or DCM, the polypeptide fragment prepared by using the NMP as a swelling solvent of the resin has a significantly higher yield. Therefore, the NMP acts as the swelling solvent for the resin.

Preferably, an activation system for the amino acid activation is selected from the group consisting of 1-hydroxybenzotriazole (HOBT)/N,N-dicyclohexylcarbodiimide (DCC), 1-hydroxy-7-aza-benzotriazole (HOAT)/DCC, HOBT/N,N′-diisopropylcarbodiimide (DIC), Oxymapure/DIC, O-(benzotriazol-1-yl)-N,N,N′,N′-tetramethyluronium hexafluorophosphate (HBTU)/N,N-diisopropylethylamine (DIPEA), and O-(benzotriazol-1-yl)-N,N,N′,N′-tetramethyluronium tetrafluoroborate (TBTU)/DIPEA.

Through experimental analysis, it can be seen that compared with the HOAT/DCC, HOBT/DIC, Oxymapure/DIC, HBTU/DIPEA, or TBTU/DIPEA, the polypeptide fragment prepared by using the HOBT/DCC as an activation system during the amino acid activation has a further improved yield. Therefore, the HOBT/DCC serves as the activation system in the amino acid activation.

In the present disclosure, the HOBT/DCC activation system is cheap and easy to use, and is conducive to rapid condensation of amino acids and resin at room temperature. This process can effectively inhibit racemization in polypeptide synthesis, resulting in a higher yield of the prepared polypeptide fragment.

Preferably, the separation and purification includes: separating a resin in the peptide resin with a lysis buffer, removing the resin by filtration, removing the lysis buffer by vacuum distillation, water dissolution, and extraction by an extractant in sequence.

Preferably, the lysis buffer includes trifluoroacetic acid (TFA), 1,2-ethancdithiol (EDT), thioanisole, and water.

Through experimental analysis, it can be seen that compared with the TFA and water, the polypeptide fragment prepared by using the TFA, EDT, thioanisole, and water as a lysis buffer has a significantly higher purity. Therefore, the TFA, EDT, thioanisole, and water act as the lysis buffer to separate the resin in the peptide resin.

In the third aspect, the present disclosure provides a polypeptide immunogen prepared using the polypeptide fragment THRB-CVD20.

In the fourth aspect, the present disclosure provides an anti-THRB polyclonal antibody prepared using the polypeptide fragment THRB-CVD20.

In the present disclosure, the anti-THRB polyclonal antibody prepared using the polypeptide fragment THRB-CVD20 has a higher titer, indicating that the anti-THRB polyclonal antibody prepared by the present disclosure has a higher sensitivity.

In the fifth aspect, the present disclosure provides use of the anti-THRB polyclonal antibody in recognition, identification, and screening of a THRB protein.

In the present disclosure, the anti-THRB polyclonal antibody prepared using the polypeptide fragment THRB-CVD20 has a high specificity and can be used to identify THRB proteins of humans, rats, and mice; the subcellular location of THRB protein indicated by the anti-THRB polyclonal antibody is accurate in rat and mouse liver tissue cells; moreover, the anti-THRB polyclonal antibody can be applied to flow cytometric sorting, and can be further used for the recognition, identification, and screening of THRB-positive cells.

In summary, this application has the following beneficial effects:

In the present disclosure, the polypeptide fragment THRB-CVD20 is prepared by the resin swelling, amino acid activation, preparation of an amino acid-resin, removal of a protecting group, preparation of a peptide resin, and separation and purification according to the amino acid sequence shown in SEQ ID NO: 1. The polypeptide fragment has high purity and yield.

In the present disclosure, screening the solvent in resin swelling and screening the activation system for amino acid activation further improve the purity and yield of the polypeptide fragment THRB-CVD20.

In the present disclosure, the anti-THRB polyclonal antibody prepared using the polypeptide fragment THRB-CVD20 as an immunogen has high sensitivity and specificity and can accurately identify human and mouse THRB proteins; the subcellular location of THRB proteins indicated by the anti-THRB polyclonal antibody is accurate in mouse liver tissue cells; moreover, the anti-THRB polyclonal antibody can be applied to flow cytometric sorting, thus enabling the recognition, identification, and screening of THRB-positive cells.

### BRIEF DESCRIPTION OF THE DRAWINGS

FIGS. 1A-1D show the high-performance liquid chromatography (HPLC) and mass spectrometry (MS) diagrams of the polypeptide fragment THRB-CVD20 prepared in Example 1 and the polypeptide fragment prepared in Comparative Example 1 (where FIG. 1A is the HPLC diagram of the polypeptide fragment THRB-CVD20 prepared in Example 1; FIG. 1B is the MS diagram of the polypeptide fragment THRB-CVD20 prepared in Example 1; FIG. 1C is the HPLC diagram of the polypeptide fragment prepared in Comparative Example 1; and FIG .1D is the MS diagram of the polypeptide fragment prepared in Comparative Example 1);

FIGS. 2A-2B show the enzyme-linked immunosorbent assay (ELISA) titer detection charts of the anti-THRB polyclonal antibody prepared in Example 10 and the polyclonal antibody prepared in Comparative Example 2 (where FIG. 2A is the ELISA titer detection chart of the anti-THRB polyclonal antibody prepared in Example 10; and FIG. 2B is the ELISA titer detection chart of the anti-THRB polyclonal antibody prepared in Comparative Example 2);

FIG. 3 shows the Western Blot detection pattern of the anti-THRB polyclonal antibody prepared in Example 10;

FIGS. 4A-4B show the immunohistochemical staining images of the anti-THRB polyclonal antibody prepared in Example 10 in rat and mouse liver tissues (where FIG. 4A is the immunohistochemical staining image of the anti-THRB polyclonal antibody prepared in Example 10 in mouse liver tissues; and FIG. 4B is the immunohistochemical staining image of the anti-THRB polyclonal antibody prepared in Example 10 in rat liver tissues); and

FIGS. 5A-5B show the flow cytometric detection results of the anti-THRB polyclonal antibody prepared in Example 10 in a positive A431 cell line and a negative THP-1 cell line (where FIG. 5A is the flow cytometric detection result of the anti-THRB polyclonal antibody prepared in Example 10 in the positive cell sample; FIG. 5B is the flow cytometric detection result of the anti-THRB polyclonal antibody prepared in Example 10 in the negative cell sample; P₁ represents the anti-THRB polyclonal antibody prepared in Example 10; P₂ represents the isotype control; P₃ represents the negative control; P₄ represents the blank control).

### DETAILED DESCRIPTION OF THE EMBODIMENTS

In the first aspect, the present disclosure provides a THRB-CVD20 polypeptide fragment, where the polypeptide fragment THRB-CVD20 has an amino acid sequence shown in SEQ ID NO: 1.

In the second aspect, the present disclosure further provides a preparation method of the polypeptide fragment THRB-CVD20, including the following steps:

S1, resin swelling: adding 10 mL to 30 mL of a solvent into 0.1 mmol of a resin to allow swelling for 10 min to 20 min at a room temperature; and cleaning the resin with the solvent and draining; where the solvent is selected from the group consisting of NMP, DMF, and DCM.

S2, amino acid activation: adding 0.5 mmol of Fmoc-amino acid into an activation system to allow activation at a room temperature for 15 min to 25 min to obtain an activated amino acid; where the activation system is selected from the group consisting of HOBT/DCC, HOAT/DCC, HOBT/DIC, Oxymapure/DIC, HBTU/DIPEA, and TBTU/DIPEA; and dosages of two components in the activation system each are 0.5 mmol.

S3, preparation of an amino acid-resin: transferring the activated amino acid into the swollen resin, adding 0.3 mL to 0.6 mL of a catalyst DMAP to allow a reaction at a room temperature for 15 min to 25 min; and washing a resulting product with DCM and MeOH in sequence, and then washing with NMP to obtain the amino acid-resin.

S4, removal of a Fmoc protecting group: adding the amino acid-resin into 3 mL to 10 mL of 20% Piperidine/DMF to allow a reaction at a room temperature for 15 min to 25 min, thereby removing the Fmoc protecting group; and washing a resulting product with DCM and MeOH in sequence, and then washing with NMP to obtain an activated amino acid-resin.

S5, preparation of a peptide resin: repeating steps S1 to step S4 to sequentially couple with activated amino acids to obtain a peptide resin; where the activated amino acids for each coupling are as follows in sequence: aspartate, glutamate, proline, leucine, phenylalanine, lysine, arginine, lysine, glutamine, lysine, tryptophan, histidine, serine, glycine, glutamine, alanine, asparagine, threonine, alanine, valine, and cysteine.

S6, separation of the peptide resin: adding a lysis buffer into the peptide resin (where 8 mL to 12 mL of the lysis buffer is added per 1 g of the peptide resin) to allow a reaction at a room temperature for 3 h; after the reaction is completed, removing the resin by filtration, and removing the lysis buffer by vacuum distillation; and subjecting a resulting peptide solution to water dissolution and extraction with cold ether to obtain a crude polypeptide fragment; where TFA, EDT, thioanisole, and H₂O in the lysis buffer are at a volume ratio of 88:(1.5-2.5):(4-6):(4-6).

S7, purification of the crude polypeptide fragment: subjecting the crude polypeptide fragment to HPLC purification, and the HPLC purification includes: chromatographic column: C₁₈ 25×250 mm; chromatograph: Waters 600, Waters, USA; mobile phase A: 0.1% TFA-H₂O; mobile phase B: 0.1% TFA-60% acetonitrile; detection wavelength: 214 nm; flow rate: 10 mL/min; elution gradient: 20% to 60% of mobile phase B within 30 min; after the elution is completed, removing the solvent under reduced pressure and conducting lyophilization to obtain the polypeptide fragment THRB-CVD20.

In the third aspect, the present disclosure provides a polypeptide immunogen prepared using the polypeptide fragment THRB-CVD20.

In the fourth aspect, the present disclosure provides an anti-THRB polyclonal antibody prepared using the polypeptide fragment THRB-CVD20.

In the fifth aspect, the present disclosure provides use of the anti-THRB polyclonal antibody in recognition, identification, and screening of a THRB protein.

Table 1 shows the raw materials used in the present disclosure and their abbreviations and sources.

Table 1 Raw materials, abbreviations and sources thereof

| | Raw | |
|---|---|---|
| | material | |
| Raw material | abbreviation | Source |
| P-hydroxymethylphenoxymethyl polyethylene | HMP resin | CsBio, |
| resin, Wang Resin | | USA |
| 9-fluorenylmethoxycarbonyl-protected amino acid | Fmoc-AA | Merck |
| N-methylpyrrolidone | NMP | Merck |
| Dichloromethane | DCM | Merck |
| Methanol | MeOH | Merck |
| Piperidine | Piperidine | Merck |
| N,N-dimethylformamide | DMF | Merck |
| Dimethylaminopyridine | DMAP | Sigma |
| 1-hydroxybenzotriazole | HOBT | Sigma |
| N,N′-dicyclohexylcarbodiimide | DCC | Sigma |
| 1-hydroxy-7-aza-benzotriazole | HOAT | Sigma |
| N,N-diisopropylcarbodiimide | DIC | Sigma |
| Ethyl 2-cyano-2-(hydroxyimino)acetate | Oxymapure | Sigma |
| O-(benzotriazol-1-yl)-N,N,N′,N′-tetrameth- | HBTU | Sigma |
| yluronium hexafluorophosphate | | |
| N,N-diisopropylethylamine | DIPEA | Sigma |
| Trifluoroacetic acid | TFA | Sigma |
| 1,2-ethanedithiol | EDT | Sigma |
| Thioanisole | / | Sigma |
| Acetonitrile | / | Merck |
| Hemocyanin KLH | / | Sigma |
| 3-maleimidobenzoic acid N-hydroxysuccinimide | MBS | Sigma |
| ester | | |

The present disclosure will be further described in detail below with reference to Examples 1 to 10, Comparative Examples 1 to 2 and performance detection tests. These Examples cannot be understood as limiting the scope of protection claimed by the present disclosure.

### EXAMPLES

### Example 1

Example 1 provided a polypeptide fragment THRB-CVD20.

The polypeptide fragment THRB-CVD20 in this example had an amino acid sequence shown in SEQ ID NO: 1.

A preparation method in this example specifically included the following steps:

S1, resin swelling: 20 mL of a solvent NMP was added into 0.1 mmol of an HMP resin to allow swelling for 15 min at a room temperature; and the resin was cleaned with the NMP and drained.

S2, amino acid activation: 0.5 mmol of Fmoc-amino acid was added with 0.5 mmol of DCC and 0.5 mmol of HOBT to allow activation at room temperature for 20 min to obtain an activated amino acid.

The Fmoc-amino acid (Fmoc-AA) had a structural formula shown in formula (1) or formula (2); the amino acid had an activation reaction formula shown in formula (3).

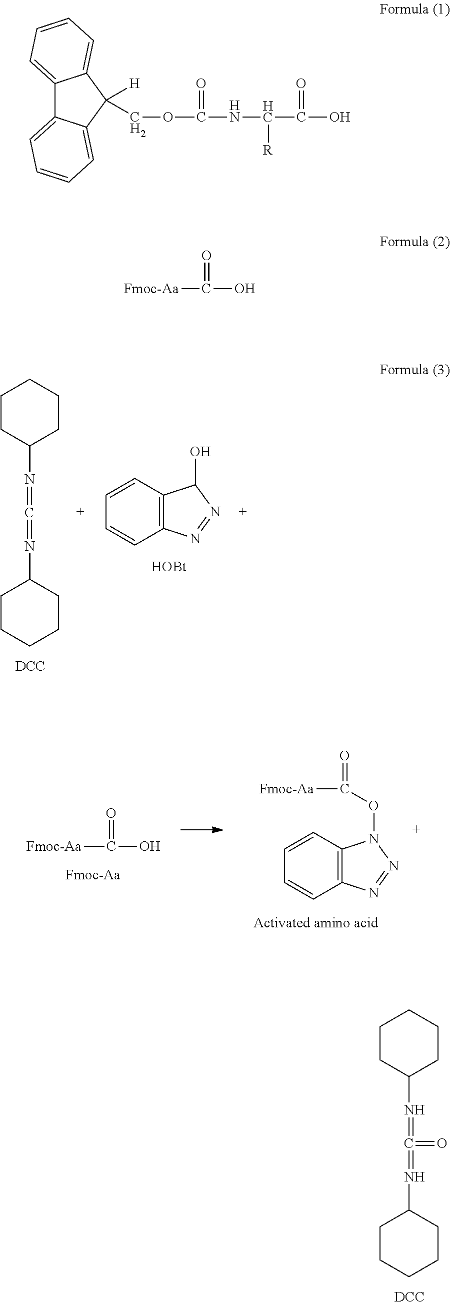

S3, preparation of an amino acid-resin: the activated amino acid was transferred into the swollen resin, 0.5 mL of a catalyst DMAP was added to allow a reaction at a room temperature for 20 min; and a resulting product was washed with DCM and MeOH in sequence 3 times, and then washed with NMP 2 times to obtain the amino acid-resin.

The amino acid-resin had a preparation reaction formula shown in formula (4).

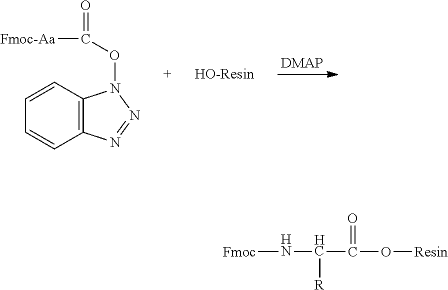

formula (4) (R represented a first amino acid ligated, Resin represented the HMP resin)

S4, removal of a Fmoc protecting group: the amino acid-resin was added into 5 mL of 20% Piperidine-containing DMF to allow a reaction at a room temperature for 20 min, thereby removing the Fmoc protecting group; and a resulting product was washed with DCM and MeOH in sequence 3 times, and then washed with NMP 2 times to obtain an activated amino acid-resin.

The removal of Fmoc protecting group had a reaction formula shown in formula (5).

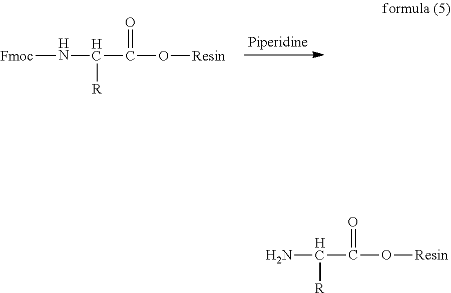

S5, preparation of a peptide resin: steps S1 to step S4 were repeated to sequentially couple with activated amino acids to obtain a peptide resin; where the activated amino acids for each coupling were as follows in sequence: aspartate, glutamate, proline, leucine, phenylalanine, lysine, arginine, lysine, glutamine, lysine, tryptophan, histidine, serine, glycine, glutamine, alanine, asparagine, threonine, alanine, valine, and cysteine.

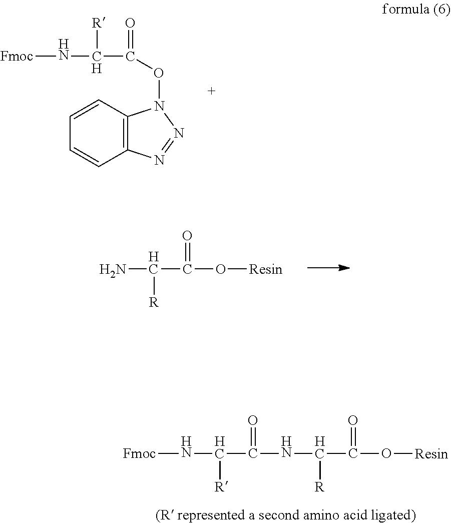

S6, separation of the peptide resin: a lysis buffer was added into the peptide resin (where 10 mL of the lysis buffer was added per 1 g of the peptide resin) to allow a reaction at a room temperature for 3 h; after the reaction was completed, the resin was removed by filtration, and the lysis buffer was removed by vacuum distillation; and a resulting peptide solution was subjected to water dissolution by 2 mL and extraction with 20 mL of cold ether to obtain a crude polypeptide fragment; where TFA, EDT, thioanisole, and H₂O in the lysis buffer are at a volume ratio of 88:2:5:5.

S7, purification of the crude polypeptide fragment: the crude polypeptide fragment was subjected to HPLC purification, and the HPLC purification included: chromatographic column: C₁₈ 25×250 mm; chromatograph: Waters 600, Waters, USA; mobile phase A: 0.1% TFA-H2O; mobile phase B: 0.1% TFA-60% acetonitrile; detection wavelength: 214 nm; flow rate: 10 mL/min; elution gradient: 20% to 60% of mobile phase B within 30 min; after the elution was completed, the solvent was removed under reduced pressure and lyophilization was conducted to obtain the polypeptide fragment THRB-CVD20.

### Example 2

This example provided a polypeptide fragment THRB-CVD20.

This example differed from Example 1 in that the solvent in resin swelling was DMF. The other steps were the same as above.

### Example 3

This example provided a polypeptide fragment THRB-CVD20.

This example differed from Example 1 in that the solvent in resin swelling was DCM. The other steps were the same as above.

### Examples 4 to 8

Examples 4 to 8 provided a polypeptide fragment THRB-CVD20 separately.

The above examples differed from Example 1 in that different activation systems were used in the amino acid activation, and a dosage of each component in the activation system was 0.5 mmol. The activation systems were specifically shown in Table 2.

Table 2 Activation systems used in amino acid activation in Examples 1 and 4 to 8

| | |
|---|---|
| Examples | Activation system in amino acid activation |

| | | |
|---|---|---|
| 1 | HOBT | DCC |
| 4 | HOAT | DCC |
| 5 | HOBT | DIC |
| 6 | Oxymapure | DIC |
| 7 | HBTU | DIPEA |
| 8 | TBTU | DIPEA |

### Example 9

Example 9 provided a polypeptide fragment THRB-CVD20.

This example differed from Example 1 in that the lysis buffer in separation of peptide resin included TFA and H₂O; the TFA and the H₂O were at a volume ratio of 88:12.

### Comparative Examples

### Comparative Example 1

This comparative example provided a polypeptide fragment.

The polypeptide fragment in this comparative example had an amino acid sequence shown in SEQ ID NO: 2.

This comparative example differed from Example 1 in that the activated amino acid for each coupling in the polypeptide fragment preparation method was: tryptophan, glutamate, glutamate, aspartate, threonine, proline, glutamate, proline, lysine, histidine, glycine, isoleucine, serine, lysine, and cysteine.

### Performance Detection Test 1

The polypeptide fragments THRB-CVD20 prepared in Examples 1 to 9 and the polypeptide fragment prepared in Comparative Example 1 were used as detection objects to detect the purity and yield of the polypeptide fragment.

Detection method: HPLC analysis was conducted to detect the purity of polypeptide fragment. Specific detection conditions included: chromatographic column: C₁₈ 4.6×150 mm; mobile phase A: 0.1% TFA-H₂O; mobile phase B: 0.1% TFA-acetonitrile; detection wavelength: 214 nm; flow rate: 1 mL/min; elution gradient: 0% to 60% of mobile phase B within 30 min.

### Detection Results

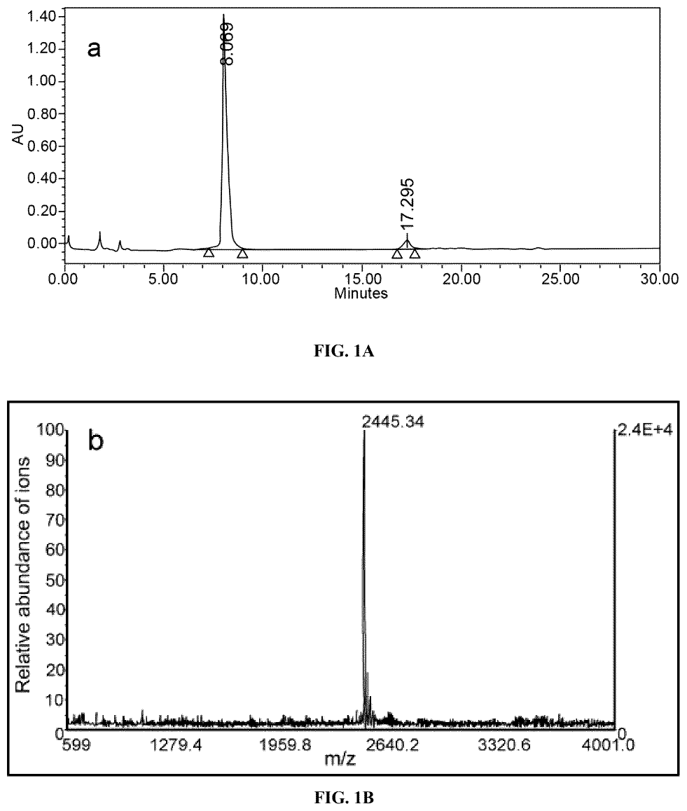

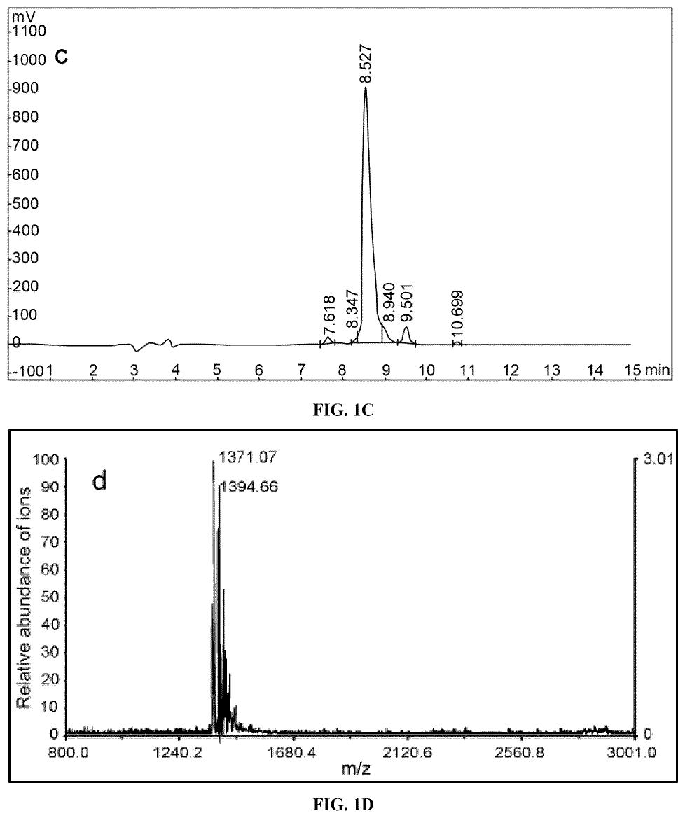

(1) FIGS. 1A-1D showed the HPLC and MS diagrams of the polypeptide fragment THRB-CVD20 provided in Example 1 and the polypeptide fragment provided in Comparative Example 1.

As shown in FIG. 1A, the polypeptide fragment THRB-CVD20 of Example 1 had a purity detection value of 95.4%; as shown in FIG. 1B, a molecular weight detection value was 2445.34. As shown in FIG. 1C, the polypeptide fragment of Comparative Example 1 had a purity detection value of 91.5%; as shown in FIG. 1D, a molecular weight detection value was 1371.97. The above detection results showed that the present disclosure successfully prepared the polypeptide fragment THRB-CVD20 with high purity.

(2) Table 3 showed the purity and yield of the polypeptide fragments prepared in Examples 1 to 9 and Comparative Example 1.

Table 3 Purity and yield of polypeptide fragments

| | | Detection results | | | | Detection results | |
|---|---|---|---|---|---|---|---|
| Detection | | Purity | Yield | Detection | | Purity | Yield |
| object | | (%) | (%) | object | | (%) | (%) |
| Example | 1 | 95.4 | 93.5 | Example | 6 | 95.3 | 90.7 |
| | 2 | 93.8 | 88.2 | | 7 | 94.8 | 89.5 |
| | 3 | 94.6 | 90.3 | | 8 | 93.6 | 91.5 |
| | 4 | 94.9 | 91.1 | | 9 | 92.1 | 92.4 |
| | 5 | 93.2 | 92.6 | / | | | |
| Comparative | 1 | 91.5 | 93.2 | | | | |
| Example | | | | | | | |

Combined with Table 3, by analyzing the detection results of Examples 1 to 9 and Comparative Example 1, the polypeptide fragment was prepared through resin swelling, amino acid activation, preparation of an amino acid-resin, removal of a protecting group, preparation of a peptide resin, and separation and purification. These polypeptide fragments had a purity of greater than 91% and a yield of greater than 88%, indicating that the polypeptide fragments prepared using the technical solutions provided by the present disclosure had higher purity and yield.

By comparing the detection results of Examples 1 to 3, it can be seen that compared to using the DMF or DCM, the polypeptide fragment prepared by using the NMP as a swelling solvent of the resin has a significantly higher yield. Therefore, the NMP acts as the swelling solvent for the resin.

By comparing the detection results of Examples 1 and 4 to 8, it can be seen that compared with the HOAT/DCC, HOBT/DIC, Oxymapure/DIC, HBTU/DIPEA, or TBTU/DIPEA, the polypeptide fragment prepared by using the HOBT/DCC as an activation system during the amino acid activation has a further improved yield. Therefore, the HOBT/DCC serves as the activation system in the amino acid activation.

By comparing the detection results of Examples 1 and 9, it can be seen that compared with the TFA and water, the polypeptide fragment prepared by using the TFA, EDT, thioanisole, and water as a lysis buffer has a significantly higher purity. Therefore, the TFA, EDT, thioanisole, and water act as the lysis buffer to separate the resin in the peptide resin.

### Example 10

This example provided an anti-THRB polyclonal antibody.

A polypeptide fragment used in this example was the polypeptide fragment THRB-CVD20 prepared in Example 1.

A preparation method in this example specifically included the following steps:

### S1: Preparation of a Polypeptide Immunogen

A borate buffer with pH=8.5 and a concentration of 50 mmol/L was prepared as a coupling buffer A; a phosphate-buffered saline (PBS) containing 0.15 mol/L NaCl and 0.01 mol/L Na2EDTA, pH=7.0, with a concentration of 0.1 mol/L was prepared as a coupling buffer B.

5 mg (0.11 μmol) of hemocyanin KLH was dissolved in 0.75 mL of the coupling buffer A to obtain a hemocyanin KLH solution; 3 mg (11 μmol) of MBS was added into 75 μL of DMF in three portions and fully dissolved to obtain an MBS solution; the hemocyanin KLH solution and the MBS solution were mixed well by vortex to allow reaction at room temperature for 30 min; and a resulting reaction mixture was eluted with a PD-10 column balanced with the coupling buffer B, and an obtained eluate was collected to obtain an MBS-KLH solution.

1.5 mg of the polypeptide fragment THRB-CVD20 prepared in Example 1 was dissolved in 0.15 mL of the coupling buffer B, added with 0.56 mL of the MBS-KLH solution, and mixed well by rotation to allow reaction at room temperature for 2 h, and placed overnight at 4° C.; a resulting reaction mixture was eluted with the PD-10 column balanced with the coupling buffer B, and an obtained eluate was collected to obtain a THRB-CVD20 polypeptide immunogen.

### S2: Preparation of an Anti-THRB Polyclonal Antibody

1 mL of Freund's complete adjuvant and 1 mL of the THRB-CVD20 polypeptide immunogen with a concentration of 1 mg/mL were thoroughly mixed to obtain a basic immunizer; 1 mL of Freund's incomplete adjuvant and 1 mL of the THRB-CVD20 polypeptide immunogen with a concentration of 1 mg/mL were thoroughly mixed to obtain a booster immunizer; where the Freund's complete adjuvant (Cat No. 344289) and the Freund's incomplete adjuvant (Cat No. 344291) were both purchased from Sigma-Aldrich.

Experimental-grade New Zealand white rabbits with a body weight of 1.5±0.5 kg were injected with the basic immunizer, and then the booster immunizer was injected multiple times at the 3rd, 5th, and 7th weeks, thus conducting multi-point immunization on the back and foot pads; on the 7th day after immunization, blood was collected from the auricular vein, serum was separated, and an antibody titer of the THRB-CVD20 was detected by ELISA; when an antibody concentration in the serum reached the peak, the white rabbit with the best detection data was selected to conduct carotid artery phlebotomy, serum was separated, and the anti-THRB polyclonal antibody was obtained through Protein A/G affinity purification.

### Comparative Example 2

This comparative example provided a polyclonal antibody.

The difference between a preparation method in this comparative example and that in Example 10 was that a polypeptide fragment used was the polypeptide fragment prepared in Comparative Example 1.

### Performance detection test 2

The anti-THRB polyclonal antibody provided in Example 10 and the polyclonal antibody provided in Comparative Example 2 were used as detection objects to detect the performance of the anti-THRB polyclonal antibody.

### (1) Titer of the Anti-THRB Polyclonal Antibody

The anti-THRB polyclonal antibody provided in Example 10 and the polyclonal antibody provided in Comparative Example 2 were used as detection objects to detect the titer of the anti-THRB polyclonal antibody.

Detection method: a sensitivity of the anti-THRB polyclonal antibody was tested according to an ELISA method specified in the Volume III of the 2020 edition of the Pharmacopoeia of the People's Republic of China to obtain the titer.

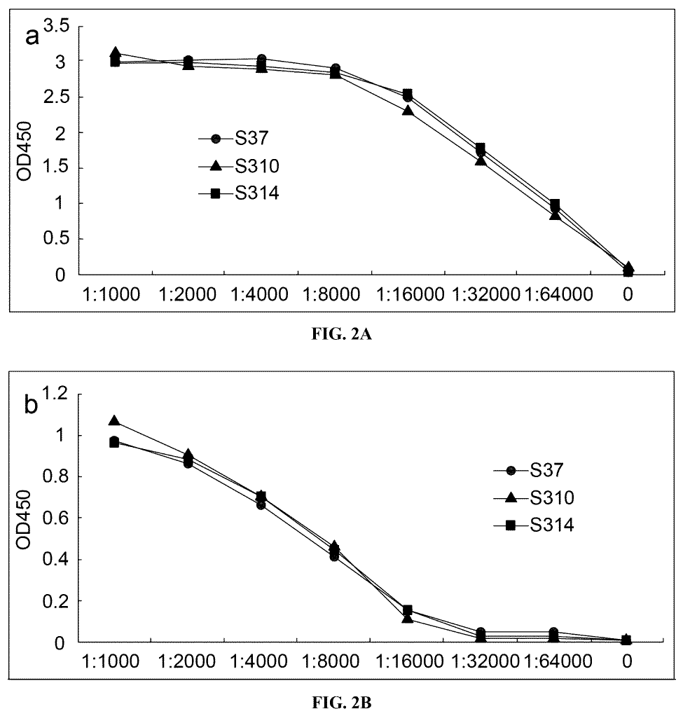

Detection results: FIGS. 2A-2B showed an ELISA titer detection chart of the anti-THRB polyclonal antibody prepared in Example 10 and the polyclonal antibody prepared in Comparative Example 2.

After analyzing the detection results of Example 10 and Comparative Example 2 in conjunction with FIGS. 2A-2B, the anti-THRB polyclonal antibody prepared in Example 10 had a titer of 1:128000, indicating that the polyclonal antibody prepared from the polypeptide fragment THRB-CVD20 prepared in Example 1 showed an extremely high sensitivity. However, the polyclonal antibody prepared in Comparative Example 2 had a titer of less than 1:16000, indicating that qualified antibodies were not obtained through the polypeptide fragment provided in Comparative Example 1.

(2) Specificity of the Anti-THRB polyclonal antibody in recognizing THRB protein

The anti-THRB polyclonal antibody provided in Example 10 was used as the detection object to detect the specificity of the anti-THRB polyclonal antibody.

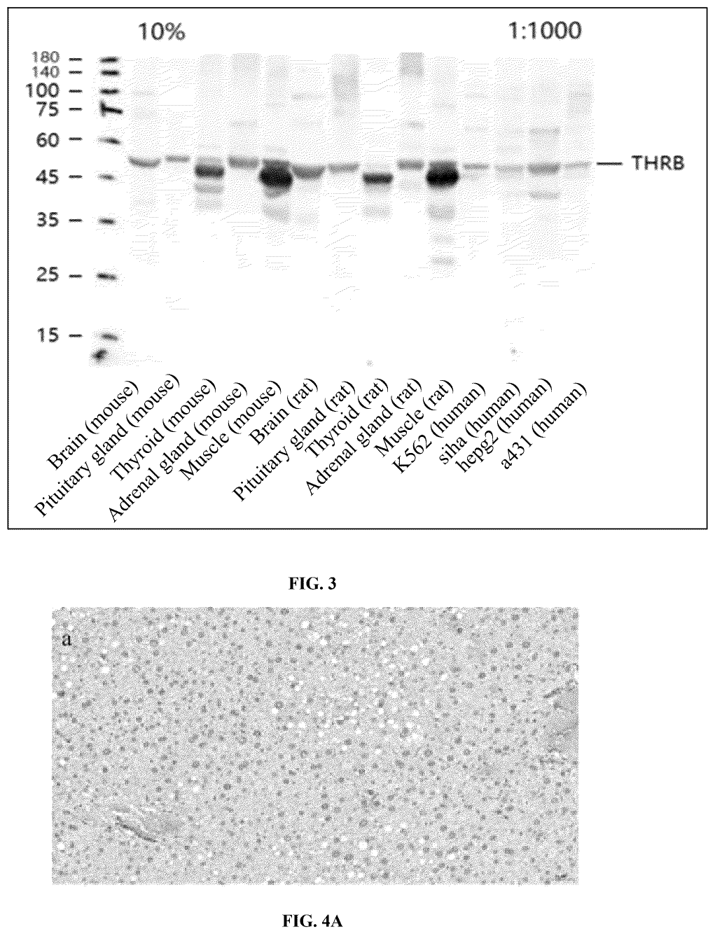

Detection method: the specificity of the anti-THRB polyclonal antibody was detected by Western blot. Specifically: different tissues from different species of humans, rats, and mice were used as samples, a total of 14 samples, as shown in FIG. 3; 4% stacking gel and 8% resolving gel were used to load the samples, the voltage was adjusted to 140 V until the samples lined up, then the minimum voltage was adjusted to 70 V, and the electrophoresis was terminated when the indicator bromophenol blue in the lysis buffer migrated out of the lower edge of the glass plate; the gel was collected for electrotransfer to a NC or PVDV membrane at 75 V for 90 min; an appropriate amount of the 200-fold diluted anti-THRB polyclonal antibody provided in Example 10 was added on the membrane, and incubated at room temperature for 2 h; the membrane was washed 3 times with TBST, 10 min each time; an appropriate amount of HRP-labeled goat anti-rabbit secondary antibody diluted 5000 times was added, and incubated at room temperature for 1 h; the membrane was washed 3 times with TBST, 10 min each time; the membrane was reacted with a chemiluminescence detection reagent in a darkroom for 3 min, then placed in a dark box covered with plastic wrap, wrapped in PVDF membrane, and then exposed to Kodak X film for 1 min in the darkroom.

Detection results were shown in FIG. 3.

Based on literature reports, the THRB protein had a molecular weight of approximately 53 kDa and a 46 kDa cleavage fragment (double band). As shown in FIG. 3, Western blot detection patterns of different samples from different species of humans, rats, and mice showed that the detection band was located at 53/46 kDa, which was exactly the same as the actual molecular weight of THRB protein reported in the literature, and had less non-specific bands. The above results showed that the anti-THRB polyclonal antibody prepared in Example 10 could recognize human, rat, and mouse THRB proteins with high specificity.

### (3) Accuracy of the Anti-THRB Polyclonal Antibody in Recognizing the Target Protein Location in Tissues

The anti-THRB polyclonal antibody provided in Example 10 was used as the detection object to detect the accuracy of the anti-THRB polyclonal antibody in recognition of target protein location in tissues.

Detection method: an expression site of the anti-THRB polyclonal antibody provided in Example 10 in cells was detected through immunohistochemistry (IHC) to determine the accuracy of the anti-THRB polyclonal antibody provided in Example 10 in recognizing the target protein location in tissues.

The detection specifically included the following steps:

S1: paraffin sections of mouse liver tissue and paraffin sections of rat liver tissue were sequentially treated by xylene I 15 min—xylene II 15 min—xylene III 15 min—absolute ethanol I 5 min-absolute ethanol II 5 min-90% ethanol 5 min—85% ethanol 5 min—washing with distilled water.

S2: the tissue sections were placed in 0.01 M, pH=6.0 citric acid repair solution, boiled for 15 min to allow thermal repair, and washed 3 times with PBS (PH=7.4) for 5 min each time; the tissue sections were placed in 3% hydrogen peroxide, incubated in the dark at room temperature for 20 min to block endogenous peroxide, and washed 3 times with PBS (PH=7.4) for 5 min each time.

S3: 3% goat serum was added dropwise to cover the tissue evenly and sealed at room temperature for 30 min; the tissue was incubated with the anti-THRB polyclonal antibody provided in Example 10 diluted 200 times as a primary antibody at 4° C. overnight; biotin-labeled goat anti-rabbit IgG and HRP-labeled streptavidin were added in sequence and incubated at room temperature for 50 min.

S4: DAB color developing solution was added dropwise to the tissue sections, and rinsed with tap water for 10 min after color development; the tissue sections were counterstained with hematoxylin for 3 min, rinsed with tap water, differentiated with hematoxylin differentiation solution for a few seconds, rinsed with tap water, the hematoxylin converted to purplish blue with “bluing” solution, and rinsed with running water; the tissue sections were immersed in the following solutions in sequence to make them dehydrated and transparent: 75% alcohol 5 min-85% ethanol 5 min-absolute ethanol I 5 min-absolute ethanol II 5 min-xylene I 5 min; the sections were taken out of xylene, air-dried briefly, and mounted with neutral gum; after air-drying, the sections were mounted with coverslip gum.

S5: after taking pictures under a microscope, the tissue sections were analyzed with ImageJ analysis software.

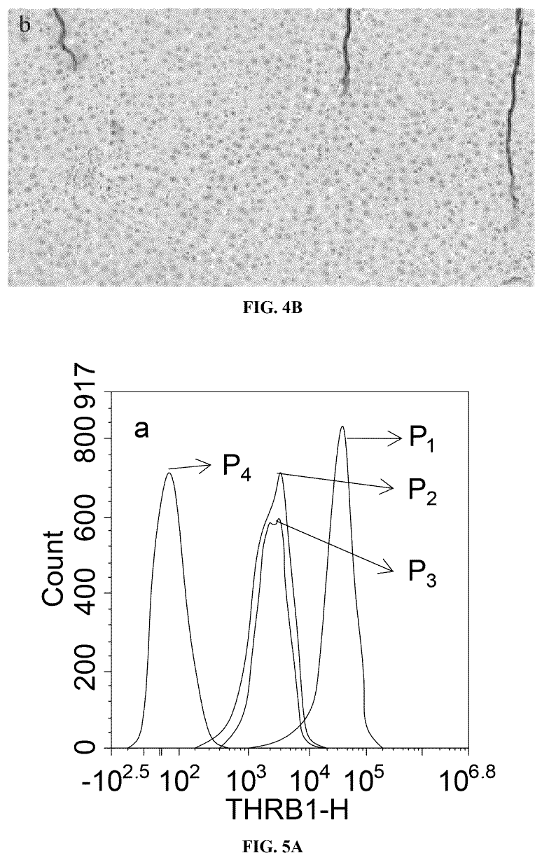

The detection results were shown in FIGS. 4A-4B; where FIG. 4A was the immunohistochemical staining image of the anti-THRB polyclonal antibody prepared in Example 10 in mouse liver tissues; and FIG. 4B was the immunohistochemical staining image of the anti-THRB polyclonal antibody prepared in Example 10 in rat liver tissues.

From the detection results in FIGS. 4A-4B, it was concluded that in rat and mouse liver tissues, consistent with the information that the THRB were mainly located in the nuclear area of secretory nerve cells, oligodendrocytes, basophils and some cancer cells. The detection results showed that the subcellular location of THRB proteins indicated by the anti-THRB polyclonal antibody prepared in the present disclosure was accurate and highly specific.

### (4) Recognition Specificity of the Anti-THRB Polyclonal Antibody for THRB Positive and Negative Living Cells

The anti-THRB polyclonal antibody provided in Example 10 was used as the detection object to detect the recognition specificity of the anti-THRB polyclonal antibody for THRB positive and negative living cells.

Detection method: the recognition specificity of the anti-THRB polyclonal antibody to THRB positive and negative living cells was detected by flow cytometry. Specific: THRB positive cell lines: K562, Siha, and A431 as well as THRB negative cell lines: H1-60, Molt-4, and ThP-1 were cultured separately. Cells were placed in 4% PFA, fixated at room temperature for 10 min, and then placed in 90% ice-cold methanol at −20° C. to permeabilize the membrane for 20 min; the cells were blocked with 5% BSA for 30 min; 1 μg of the anti-THRB polyclonal antibody prepared in Example 10 was added as a primary antibody and incubated at room temperature for 30 min; 0.5 μg of goat anti-rabbit secondary antibody IgG-AF488 was added and incubated at room temperature for 40 min. 20,000 events were acquired for analyzation by flow cytometry.

At the same time, a sample without adding the anti-THRB polyclonal antibody prepared in Example 10 was used as a blank control; a sample using normal rabbit IgG instead of the anti-THRB polyclonal antibody prepared in Example 10 was used as an isotype control; PBS with pH=7.4 instead of the anti-THRB polyclonal antibody sample prepared in Example 10 was used as a negative control.

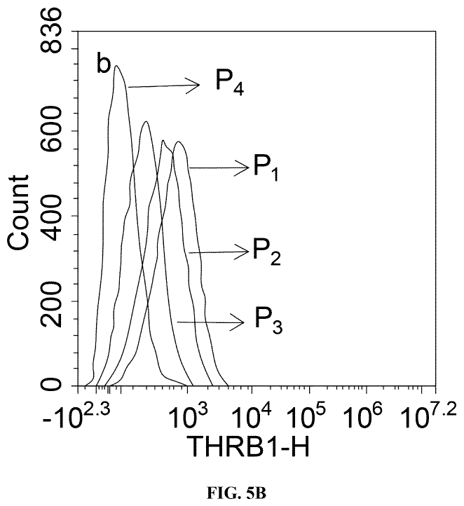

The detection results were shown in FIGS. 5A-5B; where FIG. 5A was the flow cytometric detection result of the anti-THRB polyclonal antibody prepared in Example 10 in the positive cell sample; FIG. 5B was the flow cytometric detection result of the anti-THRB polyclonal antibody prepared in Example 10 in the negative cell sample.

From the detection results in FIGS. 5A-5B, it was concluded that the anti-THRB polyclonal antibody could be detected in THRB-positive (A431 cell line), but not in THRB-negative (THP-1 cell line). This indicated that the anti-THRB polyclonal antibody prepared by the present disclosure could be applied to flow cytometric sorting with high specificity, and could be further used for the recognition, identification, and screening of THRB-positive cells.

According to the results of the above performance detection tests, it can be seen that the present disclosure has established a highly ideal method for preparing and screening immunogenic polypeptide fragments, and then successfully prepared highly-specific antibodies for flow cytometry detection. That is, according to the structure and function of the THRB protein and with the help of protein database analysis software, the solid-phase peptide synthesis method is adopted to synthesize polypeptide fragments and conjugate same to carrier proteins to complete the preparation of immunogens. After immunizing animals and screening out the best antigenic epitopes through experiments, the anti-THRB polyclonal antibody is successfully prepared with strong specificity, high sensitivity, and desirable stability. The present disclosure provides scientific researchers with high-quality antibodies for flow cytometry detection, can lay the foundation for further research on the function of THRB-positive cells, and opens up a new idea for developing flow cytometry antibodies.

Although the present disclosure has been described in detail above with general description and specific embodiments, some modifications or improvements can be made on the basis of the present disclosure, which will be apparent to those skilled in the art. Therefore, all of these modifications or improvements made without departing from the spirit of the present disclosure fall within the claimed scope of the present disclosure.

What is claimed is:

1. A polypeptide fragment thyroid hormone receptor betal (THRB)-CVD20, wherein the polypeptide fragment THRB-CVD20 has an amino acid sequence shown in SEQ ID NO: 1.

2. A preparation method of the polypeptide fragment THRB-CVD20 according to claim 1, comprising the following steps: resin swelling, amino acid activation, preparation of an amino acid-resin, removal of a protecting group, preparation of a peptide resin, and separation and purification.

3. The preparation method of the polypeptide fragment THRB-CVD20 according to claim 2, wherein a solvent for the resin swelling is selected from the group consisting of N-methylpyrrolidone (NMP), N,N-dimethylformamide (DMF), and dichloromethane (DCM).

4. The preparation method of the polypeptide fragment THRB-CVD20 according to claim 2, wherein an activation system for the amino acid activation is selected from the group consisting of 1-hydroxybenzotriazole (HOBT)/N,N′-dicyclohexylcarbodiimide (DCC), 1-hydroxy-7-aza-benzotriazole (HOAT)/DCC, HOBT/N,N-diisopropylcarbodiimide (DIC), Oxymapure/DIC, O-(benzotriazol-1-yl)-N,N,N′,N′-tetramethyluronium hexafluorophosphate (HBTU)/N,N-diisopropylethylamine (DIPEA), and O-(benzotriazol-1-yl)-N,N,N′,N′-tetramethyluronium tetrafluoroborate (TBTU)/DIPEA.

5. The preparation method of the polypeptide fragment THRB-CVD20 according to claim 2, wherein the separation and purification comprises: separating a resin in the peptide resin with a lysis buffer, removing the resin by filtration, removing the lysis buffer by vacuum distillation, water dissolution, and extraction by an extractant in sequence.

6. The preparation method of the polypeptide fragment THRB-CVD20 according to claim 5, wherein the lysis buffer comprises trifluoroacetic acid (TFA), 1,2-ethanedithiol (EDT), thioanisole, and water.

7. A polypeptide immunogen prepared using the polypeptide fragment THRB-CVD20 according to claim 1.

8. An anti-THRB polyclonal antibody prepared using the polypeptide fragment THRB-CVD20 according to claim 1.

9. A method of preparing a product for recognition, identification, and screening of a THRB protein in research, comprising using the anti-THRB polyclonal antibody according to claim 8.
